(19) United States
(12) Patent Application Publication
(10) Pub. No.: US 2009/0176208 A1
(43) Pub. Date: Jul. 9, 2009

(54) Methods for detecting, identifying and reporting the presence of animal pathological agents

(76) Inventor: Simon Brodie, San Diego, CA (US)

Correspondence Address: MANDEL & ADRIANO, SUITE 203, 572 EAST GREEN STREET, PASADENA, CA, 91101, US

(21) Appl. No.: 11/649,578

(22) Filed: Jan. 4, 2007

Related U.S. Application Data
(60) Provisional application No. 60/756,092, filed on Jan. 4, 2006.

(51) Int. Cl. C12Q 1/68

(52) U.S. Cl. 435/6

(57) ABSTRACT

A business method for use in detecting the presence of one or more pathological agents in a biological sample taken from an animal, using known diagnostic methods including antibodies and PCR, marketing test kits for obtaining and transporting biological samples from animals to a processing center, and reporting the results of diagnosis of biological samples from one or more animals.

This application claims priority to provisional patent application, U.S. Ser. No. 60/756,092, filed Jan. 4, 2006, the contents of which are hereby incorporated by reference in their entirety herein.

Throughout this application various publications are referenced. The disclosures of these publications in their entireties are hereby incorporated by reference into this application in order to more fully describe the state of the art to which this invention pertains.

### FIELD OF THE INVENTION

The present invention relates to a business system and methods, for diagnostic evaluation of biological samples from animals, more specifically to detect pathological agents, and to the reporting of the results of the evaluation in a readily accessible format that also provides a database for reference.

### BACKGROUND OF THE INVENTION

There have been a number of epidemics of animal diseases worldwide, including “mad cow disease” and “avian flu” outbreaks, requiring the destruction of large numbers of animals, at a large cost, financially and emotionally. In addition, transmission between species of animals (e.g. avian influenza virus transmitted to equines), as well as animal to human transmission of disease, has occurred. In addition, animal disease agents mutate, and may become resistant to drugs. The pathological agents associated with many diseases have been isolated, and the nucleic acid sequences determined. This provides a means for developing diagnostic and therapeutic means, including vaccines, for detecting and treating the disease.

Influenza v. is the designation for a group of orthomyxoviruses that cause influenza, including at least three genera: Influenzavirus A, Influenzavirus B, and Influenzavirus C. Antigenic variants are classified on the basis of their surface antigens (hemagglutinin and neuraminidase) as H1N1, H2N2, etc. These two surface antigens, hemagglutinin (HA) and neuraminidase (NA), are the basis of describing the serologic identity of the influenza viruses using the letters H and N with the appropriate numbers in the virus designation e.g., H1N1, H2N2, H3N8, H7N2, etc. There are at least 15 hemagglutinin and 9 neuraminidase antigens described among the Type A influenza viruses. The type designation (A, B, or C) is based upon the antigenic character of the M protein of the virus envelope and the nucleoprotein within the virus particle. All influenza viruses affecting domestic animals (equine, swine, avian) belong to Type A, and Type A influenza virus is the most common type producing serious epidemics in humans. Serotype A viruses are subject to major antigenic changes (antigenic shifts) as well as minor gradual antigenic changes (antigenic drift).

Influenza respiratory infections, particularly by Influenza A viruses, remain a serious health concern. Variations in the highly contagious Avian influenza A (“bird flu”) virus, (K. Subbarao et al., Science 279:393-396 (1998)), have resulted in serious illness, and even death, in a variety of animals including, birds, horses, pigs, canines, felines (big cats and domestic cats), and humans. This has created a concern that the virus will become pandemic, and result in many more animal and human deaths.

Recently, there have been outbreaks of severe respiratory disease in dogs, initially in racing greyhounds. Researchers isolated an influenza A virus, A/canine/Florida/43/2004, which is closely related to H3N8 equine influenza virus (Crawford et al., Science 310: 482-485 (2005); published online Sep. 26, 2005 [DOI: 10.1126/science. 1117950] (in Reports)). The geographic expansion of the infection and its persistence for several years, indicates efficient transmission of canine influenza virus among greyhounds. Recent evidence of infection in pet dogs suggests that this infection may also become enzootic in this population. Transmission of virus from one host species to another is a crucial feature of the ecology and epidemiology of influenza virus (R. G. Webster, Emerg. Infect. Dis., 4:436 (1998)).

Influenza A viruses contain two major surface glycoproteins, hemagglutinin (HA) and neuraminidase (NA) (Lamb and Krug, Fields et al., editors, Fields Virology, 3^{rd} ed., Philadelphia, Pa., Lippincott-Raven Publishers, PP. 1353-1395 (1996)). The HA protein, a trimeric type I membrane protein, is responsible for virus binding to cell surface sialyloligosaccharide receptors and for mediating fusion between the viral envelope and cellular membranes. The NA possesses enzymatic activity that cleaves α-ketosidic linkages between the terminal sialic acid and adjacent sugar residues of cellular glycoconjugates. (G M Air et al., Proteins Struct Funct Genet. 6:341-356 (1989)).

Two basic mechanisms of interspecies transmission of influenza virus are possible (R. G. Webster et al., Microbiol. Rev. 56:152 (1992), Lipatov et al., J. Virol. 78:8951 (2004)). One is the direct transfer of an essentially unaltered virus from one species to another. Examples of this mechanism include the recent human infections with the H5N1 subtype of avian influenza virus (K. Subbarao et al., Science 279:393 (1998), J. S. Peiris et al., Lancet 363:617 (2004), Y. Guan et al., Proc. Natl. Acad. Sci. USA 101:8156 (2004)). The second mechanism is a consequence of the segmented nature of the influenza genome. Simultaneous co-infection of a host with viruses from different species can result in reassortment of the segmented viral genes and the generation of a reassortant virus with the ability to infect other species. For example, novel viruses generated by gene reassortment between avian and human influenza viruses resulted in influenza pandemics in 1957 and 1968 (R. G. Webster et al., Microbiol. Rev. 56:152 (1992), Lipatov et al., J. Virol. 78:8951 (2004), Y. Kawaoka et al., J. Virol. 63:4603 (1989)). Most direct transmissions of whole influenza viruses from the natural host species to a different one do not result in sustained transmission in the new host species. Multiple virus-host interactions are necessary for replication and horizontal transmission and provide a barrier to perpetuation of influenza viruses in the new host (R. Webby et al., Nat. Med. 10:S77 (2004)). New, long-lived host-specific lineages of influenza virus, have occurred in domestic poultry, pigs, horses, and humans (R. G. Webster et al., Microbiol. Rev. 56:152 (1992), Lipatov et al., J. Virol. 78:8951 (2004)). Two clinical syndromes were evident: a milder illness characterized by initial fever and then cough for 10-14 days with subsequent recovery, or a peracute death associated with hemorrhage in the respiratory tract.

The number of animal pathological agents is quite large. Some examples of serious pathological agents in felines include, but are not limited to, Bartonella henselae, Borrelia burgdorferi, Chlamydia psittaci, Dirofilaria immitis, Ehrlichia canis, Feline Calicivirus, Feline Coronavirus (FCoV), Feline Herpesvirus 1, Feline Immunodeficiency Virus, Feline Leukemia Virus, Leptospira spp, Mycoplasma haemofelis (large form), Panleukopenia Virus, Toxoplasma gondii, and West Nile Virus.

Canine pathogens include, but are not limited to, Canine Adenovirus, Canine Distemper Virus, Canine Herpesvirus, Bordetella bronchiseptica, Neospora Hughesi and Caninum, Anaplasma phagocytophilum, Rickettsia rickettsii (RMSF), Anaplasma platys, Canine parainfluenza virus, Tritrichomonas foetus, Clostridium difficle, Cryptosporidium spp., Cryptosporidium felis, Pan Fungal and Mycobacterium spp., Salmonella spp., Choroidal Hypoplasia, Congenital stationary night blindness, Progressive Retinal Atrophy, Cystinuria, Canine Leukocyte Adhesion Deficiency, PKA, Giardia spp and Taenia spp.

Equine pathogens include, but are not limited to, Equine Herpes Virus # 1, Equine Influenza A, Lawsonia intracellularis, Streptococcus equi, Equine Arteritis virus, Equine Herpes Virus # 4, Campylobacter jejuni, E. Coli 0157:H7, EPM, Shigella spp., Yersinia enterocolitica, Rhodococcus equi, West Nile and Leptospira spp.

Marine mammal pathogens include, but are not limited to, bacteria: Staph sp., Strep sp., Erysipelas rhusiopathiae, Bartonella, Coxiella, Chlamydia, Pseudomonas sp., Pseudomonas pseudomallei, Pseudomonas mallei, Klebsiella, E. coli, Salmonella sp., Clostridia perfringens and Enterococcus; viruses: Dolphin pox, seal pox 1 and 2, papilloma universal, papilloma manatee, canine adenovirus, influenza A and B, hepatitis A and B, Bovine enterovirus, Cosackivirus, encephalomyocarditis virus, WNV SLE VEE St. Louis, Morbilliviruses: PMV, DMV, canine distemper virus, Bovine corona virus, Bovine rotavirus, universal herpes and echovirus; fungi: Aspergillus, Nocardia, Histoplasma, Blastomyces, Coccidioides immitis, Lacazia loboi, Saksenaea and Aphophysomyces.

There is thus a need for rapid, accurate and affordable methods to detect the presence of pathogens, such as influenza A viruses, and variations, in animals and humans. Early detection will result in a reduction in the costs of treatment and fatalities, in part because infected individuals can be rapidly identified, isolated to prevent spread of the virus and treated.

Methods are known for detecting the presence of pathological agents in a sample from a human or animal.

Traditionally, virus isolation, followed by HA and NA subtyping, has been the standard method, to detect and subtype influenza A viruses. A standard diagnostic test for influenza A virus uses allantoic fluid to evaluate the presence of hemagglutinating activity (Burleson et al., Virology: A Laboratory Manual (Academic Press, 1992); Kendal et al., Concepts and Procedures for Laboratory-based Influenza Surveillance, Kendal et al., Eds. (U.S. Dept. Health and Human Services, Centers for Disease Control (CDC), Atlanta, Ga., 1982)). Hemagglutination is the clumping of red blood cells that can be caused by viruses. This property is used to identify antibodies in a sample of the subject's blood. Red blood cells (RBCs) are coated with an antigenic portion of a pathogenic agent and incubated with serum from a test subject. If the serum contains antibodies to the pathogenic agent, the cells will clump (hemagglutinate). The antigenic subtype of the HA and NA surface proteins is used to subtype the virus. The most commonly used diagnostic test for subtyping is the hemagglutinating inhibition (HI) test, to quantitate serum antibody to a specific pathogenic agent, using antisera prepared against the 15 different HA subtypes. Antibodies against the virus will inhibit hemagglutination of red blood cells (RBCs). HA antigen is added to each test container. A portion of a biological sample serum is then placed in a first container, and serially diluted. After incubation, chicken RBCs are added to each container. If antibody is present in the biological sample, indicating the presence of a pathogenic agent, the RBCs will not agglutinate in response to the HA antigen.

Detection methods include antibody based assays, such as enzyme immunoassays (EIAs), including ELISA assays (Jin et al., Avian Dis. 48(4):870-8 (2004)), which use an antibody that recognizes the pathogen of interest in the sample. Such assays are available at veterinary diagnostic laboratories and commercially (FlockChek® Avian Influenza Antibody Test Kit, Idexx Laboratories, Westbrook, Me.) Typically such assays require that the pathological agent have been present in the host for sufficient time to cause the production of antibodies. In addition, antibodies made by a subject's immune system in response to infection by a pathological agent, can be detected using known methods such as hemagglutinating and inhibition of hemagglutinating activity (HI) assays. This provides the ability to detect the previous exposure of a subject to a pathological agent, after it may no longer be present, e.g. in the form shedding virus, in the host.

Combinations of various assays may be used to increase the sensitivity of assays for various animal pathogenic agents. (Rowe et al., J. Clin. Microbiol. 37(4):937-43 (1999)).

In addition, molecular diagnostics such as PCR, RT-PCR and oligonucleotide-based microarrays, have been developed to aid in the detection of human influenza A viruses (Fouchier et al., J. Clin. Microbiol. 38(11):4096-4101 (2000)), Maertzdorf et al., J. Clin. Microbiol. 42(3):981-6 (2004), J. Li et al., J. Clin. Microbiol. 39:696-704 (2001)), and equine type A influenza (S. Sengupta et al., J. Clin. Microbiol. 41:4542-4550 (2003), all incorporated by reference in their entirety herein) In addition, high throughput technologies include those developed by High Throughput Genomics, Inc., Tucson, Ariz., disclosed in U.S. Pat. Nos. 6,232,066, 6,238,869, 6,331,441 and 6,458,533, all incorporated by reference, in their entirety, herein. Such methods permit early detection of infection, perhaps before the subject's immunological system can mount a defense by producing antibodies.

The internet provides a resource for centralized reporting of data obtained from testing of large numbers of subject, easily accessible by veterinarians, officials and consumers.

Efficient, economic methods for rapid and sensitive detection, and reporting of the presence of pathological agents associated with diseases, have not been developed for wide distribution, for example to veterinary practitioners or animal caretakers, including research facilities and zoos.

### BRIEF SUMMARY OF THE INVENTION

Accordingly, a business method of the invention is provided, that includes the steps of collecting a biological sample from a species of animal, and analyzing the biological sample using one or more assays, including antibody based assays, and high throughput molecular assays, to identify the presence or absence of a pathological agent, for example in the form of nucleic acid, e.g. mRNA or DNA, or to detect antibodies to a pathological agent, present in the samples. The amount of pathological agent present in the samples is compared to the amount in known controls, consisting of biological samples from the animal species known to either lack or possess the pathological agent, and reporting the results of the assay on each animal tested. Diagnostic products using the assays, are marketed, for example, to veterinarians, in the form of a test kit. Databases are created from the information generated by diagnosis of samples from one or more animals, to assist in identifying the extent and geographical location of outbreaks of disease.

### DETAILED DESCRIPTION OF THE INVENTION

The methods of the invention utilize and apply a method that is able to detect the presence of pathological agents, in a biological sample from an animal, and to collect, assess and report the results of detection of the pathological agent, in one or more animals, for mass dissemination. The method of detection may be any known method for detecting the presence of a protein, or portion thereof , or amounts of nucleic acids corresponding to multiple distinct animal pathological agents in a single, small biological sample from an animal, and/or mutations of an animal pathological agent, with reliability, reproducibility, and sensitivity, in an economical format that can be readily disseminated, e.g. via the internet. In one embodiment, the system relies on sample analysis in a microarray or multiplex format for high throughput analysis. The resulting data, including the amount of an animal pathological agent present in the sample, are useful to diagnose disease in an animal. In turn, this information can provide the ability for prognosis of an animal based on distinguishing levels of pathological agent present over time, can assist in selection of therapeutic agents and treatments for the animal at various time points, and can assist in monitoring therapy. The system also provides the ability to establish the absence of pathological agents, i.e. “wellness” in an animal tested.

The reporting system of the method of the invention permits assessment of an initial outbreak in a species of animal, and the extent of a given pathogen in a geographical population of animals. This information permits the isolation of infected animals, to limit the spread of disease between animals of that species, and interspecies transmission, as well as animal to human transmission, and permits early intervention for treatment options. In addition, having this information available can provide direction to medical personnel for preparing to treat large numbers of ill animals, and humans, including assembling medical supplies, and, if available, vaccines.

The method of the invention involves the collection of a biological sample from a test animal. The sample may be any biological material, including, cells, tissue, blood, urine, sputum, sperm, hair or feces. The animal test sample may come from an animal suspected of carrying a pathological agent, whether or not exhibiting symptoms of the disease, or an animal believed to be healthy and free of such disease agent, for example as part of a periodic wellness screen. Control samples are also collected from animals known to be free of the pathological agent. Additional controls may be provided, to reduce false positive and false negative results, and verify that the reagents in the assay are actively detecting the selected pathogen or pathogens.

In addition to detecting the presence or absence of a pathological agent in a biological sample, the methods of detection used in the invention can reveal mutations in a pathological agent, such as changes in nucleic acid, resulting from the environment, drug treatment, genetic manipulations or mutations, injury, change in diet, aging, or any other characteristic(s) of a single animal. Mutations may also cause a pathological agent, e.g. a virus, to become resistant to a drug that was formerly effective, or to enable the virus to infect and propagate in a different species of animal, or human.

Pathological agents, and mutations in such agents, are detected in the form of nucleic acid, DNA or RNA, proteins, antibodies, or detectable portions thereof, associated with a complaint, illness or disease, that can be detected by known methods, including tests for detecting antibodies to a pathological agent, cell culture, and molecular diagnostics such as PCR, RT-PCR, nuclease protection assays, and high throughput genomics. The agents include, but are not limited to, parasites, bacterial infections, lyme disease, west nile, leukemia, viruses, cancer, cardiovascular disease, inflammatory disease, and infectious disease. Control samples are obtained from individuals who do not exhibit the pathological state, or positive controls known to contain the pathological agent.

The animal test sample is then assayed using the selected method. Recent developments in high throughput diagnosis permit the use of high throughput genomics assays as described in U.S. Pat. Nos. 6,232,066, 6,238,869, 6,331,441 and 6,458,533, or U.S. Pat. No. 6,733,977, all of which are incorporated by reference, in their entirety, herein.

The sample is then assayed to identify, for example, nucleic acid associated with pathological agents present in the animal test sample. The nucleic acid may be in the form of DNA or RNA. The method of the invention takes advantage of the presence of (or absence of) nucleic acid DNA or RNA, that corresponds to an animal pathological agent, or antibodies to the pathological agent, formed by the animal's immunological system.

The selected method is used to identify the presence of pathology-associated agents in a biological sample from an animal, and compared with control samples. By identifying pathological agents in an animal sample, the methods of the invention provide detection of the presence of a pathology, and permit ongoing monitoring of the progress of the pathology, or treatment of the pathology, in the animal.

Compositions, devices and methods for conducting multiple biological assays concurrently, using repeated arrays of probes specific for selected target molecules and detecting the interaction of a target molecule with a given probe, are known. These methods provide the ability to determine whether a given target is present or absent in a sample. These methods allow for high throughput analysis of multiple samples to be screened in a diagnostic assay. This provides the ability to provide rapid results for a number of pathological agents, and/or mutations at a favorable economical price.

### Sample Collection

A sample of biological material is taken from an animal. The amount and nature of the sample for testing will vary, depending on the pathogenic condition associated with the pathological agent, and the time after infection. Symptoms and titers of the pathological agent will vary for different agents and their associated diseases, and are known in the art.

In an embodiment for detecting upper respiratory infectious agents in an animal, diagnosis is facilitated by the collection of high-quality specimens, their rapid transport to the testing facility and appropriate storage before laboratory testing. Virus is best detected in specimens containing infected cells and secretions. Specimens for the direct detection of viral antigens or nucleic acids and virus isolation in cell cultures should be taken preferably, during the first 3 days after onset of clinical symptoms. A number of types of specimens are suitable to diagnose virus infections of the upper respiratory tract, including nasal swab, nasopharyngeal swab, nasopharyngeal aspirate, nasal wash and throat swabs. In addition to swabs, samples of tissue or serum may be taken, and invasive procedures, can also be performed.

Respiratory specimens should be collected and transported in 1-5 ml of virus transport media. A number of media that are satisfactory for the recovery of a wide variety of viruses are commercially available. Clinical specimens are added to transport medium. Nasal or nasopharyngeal swabs can also be transported in the virus transport medium. An example would be 10 gm of veal infusion broth and 2 gm of bovine albumin fraction V, added to sterile distilled water to 400 m. Antibiotics such as 0.8 ml gentamicin sulfate solution (50 mg/ml) and 3.2 ml amphotericin B (250 μg/ml) may be added. The medium is preferably sterilized by filtration. Nasal washes such as sterile saline (0.85% NaCl) may also be used to collect specimens of respiratory viruses.

Sera may be collected in an amount of from 1-5 ml of whole blood from an acute-phase animal, soon after the onset of clinical symptoms, preferably not later than 7 days. A convalescent-phase serum specimen may be collected, for example 14 days after onset of symptoms. Thereafter, serum specimens can be useful for detecting antibodies against respiratory viruses in a neutralization test.

Biological samples are obtained using well known methods. Blood samples may be taken, for example by jugular venipuncture from the test animal. Serum is then harvested and may be stored at −80° C. Mucosal samples may be taken using cotton swabs, to wipe the surface of the nasal or oropharyngeal passage, mouth or anus. Other types of samples may be taken as needed, such as urine and fecal samples. Typically, at least 1 ml of biological fluid will be obtained from a test animal. However, for subsequent diagnosis, smaller amounts may be used. For example, for molecular diagnostics, as little as a drop of fluid can be used, from the original sample taken from the animal.

A biological sample includes, but is not limited to, serum, plasma, whole blood, nipple aspirate, lung lavage, sputum, sperm, urine, cerebrospinal fluid, saliva, feces, sweat and tears.

In some instances, samples may be collected from individual animals over a period of time (e.g., once a day, once a week, once a month, biannually or annually). Obtaining numerous samples from an individual animal, over a period of time, can be used to verify results from earlier detections, track the progress of disease, and/or to identify response or resistance to a specific treatment, e.g. a selected therapeutic drug.

### Test Kits

A test kit is provided in the invention, for collecting a biological sample from an animal, and transporting the sample to a processing facility, for analyzing the sample and compiling the results. The sample test kits of the invention include components to obtain and store a biological sample, from an animal, for testing, using the methods of the invention, and instructions for use, as well as precautions and contact information. Such components include, but are not limited to, sterile swabs, test tubes or collection substrates, and various media for preserving the sample, until testing.

Where blood or tissue or cells are taken for sampling, the kits may include a device for safely drawing a small sample of blood from an animal, or removing a small “biopsy” for testing. If necessary, instructions for storing the samples in appropriate temperatures, for example at freezing temperature, will be provided. Typically the kits will include directions and materials for taking a sample, and sending it for processing (testing), for example to a central testing facility. Information for accessing the results, as well as collected results in databases, will also be provided in the kits.

An embodiment of the kit of the invention includes, a swab for sample collection, for example a cellulose fiber swab, and at least one test tube or sample vial containing transport medium for transferring the sample to the processing facility. In one embodiment, the test tube is labeled, for example with a two dimensional barcode, for automated sample tracking to report results, and inventory control. Kits may be sold in cases containing multiple kits.

### Detection of the Presence of Pathological Agent(s)

The methods used to detect pathological agents in a test sample from an animal, may detect the presence of one or more pathological agents in a sample, and the level of each pathological agent. Any method for detecting the pathological agent may be used, including, but not limited to, antibody assays including enzyme-linked immunosorbent assays (ELISAs), indirect fluorescent antibody (IFA) tests, hemagglutinating, and inhibition of hemagglutination (HI) assays, and Western Blot. Known cell-culture methods may be used. Positive cultures can be further identified using immunofluorescence of cell cultures or HI assay of the cell culture medium (supernatant).

In addition, methods for detecting nucleic acid (DNA or RNA) or protein, may be used. Such methods include, but are not limited to, polymerase chain reaction (PCR), and reverse transcriptase (RT) PCR tests and real time tests, and quantitative nuclease protection assays. There are commercially available test kits available to perform these assays. For example, QIAGEN (Valencia, Calif.) sells a one-step RT-PCR kit, and viral RNA extraction kit.

Primer sets specific for the hemagglutinin (HA) gene of many of the circulating influenza viruses are known, and are continually being developed. The influenza virus genome is single-stranded RNA, and a DNA copy (cDNA) must be made using a reverse transcriptase (RT) polymerase. The amplification of the RNA genome, for example using RT-PCR, requires a pair of oligonucleotide primers, typically designed on the basis of the known HA sequence of influenza A subtypes and of neurominadase (NM)-1. The primers will specifically amplify RNA of only one virus subtype. DNAs generated by using subtype-specific primers can be further analyzed by molecular genetic techniques such as sequencing. The test is preferably run with a positive control, or products are confirmed by sequencing and comparison with sequences in deposited databases. The absence of the target PCR products (i.e. a “negative” result) may not rule out the presence of the virus. Results can then be made available within a few hours, from either clinical swabs, or infected cell cultures.

In one embodiment, the presence of a pathological agent is detected by determining the presence or absence of antibodies against the agent, in a biological sample. It can take some time (e.g. weeks or months) after becoming infected, before antibodies can be detected in a blood test. Once formed, antibodies usually persist for many years, even after successful treatment of the disease. Thus, finding antibodies to a given pathological agent, e.g. Lyme disease bacteria, may not indicate whether the infection was recent or sometime in the past.

Antibody tests include enzyme-linked immunosorbent assays (ELISAs), indirect fluorescent antibody (IFA) tests, and Western Blot. Preferably, antibody testing is done using multiple tests, for example ELISA or IFA followed by Western blot.

Antibody testing should be done in a two-step process, using either the ELISA or IFA followed by the Western blot test. ELISA is considered a more reliable and accurate test than IFA, but IFA may be used if ELISA is not available. The Western blot test (which is a more specific test) should be done in all people who have tested positive or borderline positive (equivocal) in an ELISA or IFA test.

Hemagglutination activity may be detected in a biological sample from an animal, using chicken or turkey red blood cells as described (Burleson et al., Virology: A Laboratory Manual (Academic Press, 1992) and Kendal et al., in Concepts and Procedures for Laboratory based Influenza Surveillance, and Kendal et al., Eds. (U.S. Department of Health and Human Services, Centers for Disease Control and Prevention and Pan-American Health Organization, Atlanta, Ga., United States, 1982) pp. B17-B35.), incorporated by reference in their entirety herein.

In another embodiment, the presence of a pathological agent is detected by determining the presence or absence of genetic material (DNA) of the selected pathological agent from a biological sample, using polymerase chain reaction (PCR), or real time RT-PCR, techniques. PCR is used to detect the genetic material to identify a current (active) infection, for example early on, before antibodies have been formed. PCR can detect genetic material in various biological samples including, blood, stool, respiratory secretions or body tissue. Amplifying a second genetic region can further increase the specificity of PCR. Primers, which are the key pieces for a PCR test, may be publicly available or can be prepared using known methods. Preferably, both positive and negative controls are used, because negative results don't necessarily indicate that the pathological agent is not present in a subject (false negative). Examples of negative controls, include controls for the extraction procedure and water control for the PCR run. It is also desirable to confirm positive results to avoid “false positives” in which the presence of the pathological agent is indicated in error. Positive controls include a control for extraction and PCR. In addition, the sample can be “spiked” with a weak positive control in order to detect any PCR inhibitory substances that would interfere with the test. PCR and RT-PCR tests for influenza A virus are described by Fouchier et al., in the Journal of Clinical Microbiology, 38 (11):4096-4101 (2000)), and Maertzdorf et al., in Clin Microbiol. 42(3):981-986 (2004)), both of which are incorporated by reference in their entireties, herein.

In another embodiment of the invention, high throughput genomic methods are used to detect multiple target pathological agents that may be present in a biological sample from an animal. These procedures typically use a multiple-well microtiter plate, containing multiple different oligonucleotide probes specific for multiple target agents (nucleic acid: DNA or RNA, or protein) in each well, that may or may not be present in the biological sample, where the probes are attached to the surface of each well. The ability to test several targets simultaneously is known as “multiplexing.” The assays are performed using reagents and conditions effective for reaction of the probe with its respective target molecule. High Throughput methods are known in the art, for example, as described in issued U.S. Pat. Nos. 6,232,066, 6,238,869, 6,331,441 and 6,458,533, incorporated by reference in their entirety, herein, and are commercially available (e.g. High Throughput Genomics, Tucson, Ariz.). In the methods of the invention, a high throughput assay can be run using multiple (e.g. 100) plates with “wells” for containing the reactions, such as 96-well microplates, simultaneously. Each well of a plate can have multiple, different tests performed in it, by using an array of corresponding probes. For example, 100 plates, with 96 wells per plate, and each with 16 tests per well, can be used. In this case, each of 9,600 different drug candidates can be tested simultaneously, for 36 different parameters or assays. High throughput assays provide much more information for each biological sample, than do assays which test only one target pathological agent at a time. Thus, it is possible in a single initial high throughput screening assay to determine whether a sample from an animal contains any of several, serious pathological agents.

In one embodiment of the invention, a high throughput method is used, that detects messenger RNA (mRNA) corresponding to target pathological agents, and does not involve any RNA extraction, amplification, purification or biosynthetic steps. This method is known as the “quantitative nuclease protection assay or “qNPA,” (High Throughput Genomics, Inc., Tucson, Ariz.), that can quantitatively measure mRNA, from samples of fewer than 1,000 cells, without extraction or amplification (U.S. Pat. No. 6,238,869, incorporated by reference herein). In essence, the qNPA produces a stoichiometric amount of the specific nuclease protection probe for each gene, or a quantitative amount of a chemical mirror image. All the reagents that bind to the plate are synthetic and structurally unaltered by the assay. Assays can be conducted using a microplate washer, incubator and standard pipetting station. Standard automation and workstations perform all assay steps. Assay results are detected using known imaging devices, such as the Omix Imager™ (HTG, Tucson, Ariz.).

Other methods, including improvements to known methods, and newly developed methods, for rapidly and specifically detecting one or more pathological agents in a biological sample, can be used in the business method of the invention.

### Collection and Reporting of Results

On receipt of a kit at the processing facility, the test tubes containing the biological material, for example on a swab, are processed as described above. Where the test tubes are labeled or encoded, the tubes are initially scanned to read the identifying sample code, for example using a bar code scanning device to detect the bar code placed on each test tube. The identifying code is then stored and used to track each sample, during the diagnostic process, through reporting the results of the diagnostic test, and thereafter. Thus, a positive result for the presence of a given pathogen is able to be reliably correlated with the animal from which the sample was obtained.

The veterinarian or other persons, who obtained a sample to detect for the presence of pathogens in an animal, can obtain the results of the test for that animal, for example within 24 hours, via telephone, facsimile, or other forms of electronic transmission.

The dissemination of the information obtained from diagnosis of an animal sample, or multiple animal samples, can take a number of forms. Where a veterinarian's office, clinic or hospital, is the location for taking a sample from an animal, using the sample testing kits of the invention, then the results of the assay of that animal's sample, will be reported in a form accessible by the veterinarian or designated personnel. For example, the levels of a particular pathological agent will be entered into a file on a computer, that can be accessed via the internet, by a means of a number code for that sample. This provides the individual animal owner, through their veterinarian, with the results of a test for the presence or absence of pathological agents in the owner's animal. The results for individual animals tested using the methods of the invention, can also be transmitted verbally, for example by telephone or in person, or in written form, by facsimile, letter or email, or by electronic means.

### Internet Reporting

The use of the internet permits rapid reporting to veterinarians, regarding the results of diagnostic tests. The results are entered in a file that is then made available at, for example, a website on the internet. Automated programs may be used that email or send via facsimile, completed test results to veterinarians. Results from individual animals, multiple animals may be collected and retrieved via these programs. These programs can be used to send results on a pre-established, periodic basis. Additional information may also be provided on a website, for example current news stories regarding selected pathological agents, or technical documents collected for review.

### Collection of Results for Forming an Informational Database

The results of diagnostic processing of each sample from an animal, can be collected to form an informational database, consisting of results from testing of a number of animals for the pathogens selected for each test. The results may be grouped or categorized with respect to various factors, including, but not limited to, geographical location, time of detection, and length of duration of positive results for a given pathogen using a diagnostic method. One software system available for forming such a database is a SQL server database.

The internet provides access to the database of test results from multiple animals for multiple pathogens, collected according to the business methods of the invention. Individual veterinarians can access test results for an animal in their care, by “logging onto” (entering) a “website,”, and can access results, for example in different geographical locations across the United States, using the internet. In addition, researchers and government officials, seeking to identify potential epidemics and pandemics, can access the database.

In addition, the results of testing of individual animals using the methods of the invention can be stored, and used to determine patterns of disease occurrence and reoccurrence in animals for a given disease, for example across the United States.

The identification of genetic variations in pathological agents can be similarly stored, and accessed to provide a database of variations for further research and development of diagnostic and therapeutic agents and methods, and/or to predict animals susceptible to develop a pathology, or to become resistant to drugs used to treat a pathological condition.

### Inventory Management

The identifying code for each sample taken from an animal, provides the ability to determine how many test kits are purchased, and where they are located at any given time. This permits determination of all existing inventory located with veterinarians, and permits automatic reorders. In addition, a veterinary clinic or hospital will have ready access to information including numbers of tests ordered, used and historical data regarding particular animals in their care.

### Revenue Generation

In the short term, the business methods disclosed herein generate revenue by providing results of testing individual animals for the presence of pathological agents, to the animal owner, via their veterinarian. The veterinarian orders and pays for a test kit, and in turn is paid by the animal owner for use of the test kit, on a particular animal. The business methods herein can obtain revenue by various additional means, which may vary over time. Such sources may include direct sale revenue of sample testing kits, license fees to third parties, database subscription fees, and downstream royalties from various sources, including government agencies, academic institutions and universities, biotechnology and pharmaceutical companies, insurance companies, and veterinary care providers. Moreover, the results obtained from testing of multiple animal samples provides proprietary databases that can be licensed, for example to veterinary biotechnology and pharmaceutical companies, to “target” geographic locations for certain drugs where higher incidences of a given pathology are occurring, as well as new genetic variations in a pathological agent requiring the development of more specific drugs (“new drug discovery”).

It is to be understood, that the above embodiments are illustrative, and not restrictive. The scope of the invention should be determined with respect to the scope of the appended claims, along with their full scope of equivalents.

What is claimed is:

1. A business method for detecting and reporting the presence of at least one pathological agent in a biological sample from an animal, comprising:
a) disseminating means for detecting one or more pathological agent in a biological sample from an animal;
b) testing a biological sample from an animal, for the presence of one or more pathological agents; and
c) providing the results of the testing in step b) in an accessible format.

2. The method of claim 1, wherein said testing step consists of conducting an assay to detect nucleic acid corresponding to at least one pathological agent, in the biological sample.

3. The method of claim 1, wherein said testing step consists of a molecular detection method.

4. The method of claim 3, wherein said testing step consists of PCR, RT-PCR or high throughput analysis.

5. The method of claim 1, wherein said testing step consists of conducting an assay to detect in the biological sample, antibodies that recognize at least one pathological agent.

6. The method of claim 1, wherein said testing step consists of conducting an assay to detect the ability of the biological sample to inhibit hemagglutination.

7. The method of claim 1, further comprising collecting the results of testing from a number of animals into an accessible database.

8. The database of claim 7.

9. The method of claim 1, wherein the step of providing the results of the testing, comprises providing the results on the internet.

10. The method of claim 1, wherein the biological sample is selected from the group consisting of serum, plasma, whole blood, nasal aspirate, nipple aspirate, lung lavage, sputum, sperm, urine, cerebrospinal fluid, saliva, feces, sweat and tears.
